(19) United States
(12) Patent Application Publication
(10) Pub. No.: US 2025/0122575 A1
(43) Pub. Date: Apr. 17, 2025

(54) SEQUENCE PROCESS VALIDATION METHODS AND COMPOSITIONS

(71) Applicant: Freenome Holdings, Inc., South San Francisco, CA (US)

(72) Inventors: Kaitlyn COIL, South San Francisco, CA (US); Greg HOGAN, San Francisco, CA (US); Phuong MENCHAVEZ, Pacifica, CA (US); Michael PEARSON, Santa Cruz, CA (US); Hanna WEISENBERGER, San Francisco, CA (US)

(21) Appl. No.: 18/975,552

(22) Filed: Dec. 10, 2024

Related U.S. Application Data
(63) Continuation of application No. PCT/US2023/068314, filed on Jun. 12, 2023.
(60) Provisional application No. 63/351,598, filed on Jun. 13, 2022.

(51) Int. Cl. C12Q 1/6883; C12Q 1/6806; C12Q 1/6874

(52) U.S. Cl. CPC C12Q 1/6883; C12Q 1/6806; C12Q 1/6874; C12Q 2600/154; C12Q 2600/166

(57) ABSTRACT

The present disclosure provides methods and systems for preparing and using contrived nucleic acid samples for process validation and control. The contrived nucleic acid samples have prescribed specific physical and chemical characteristics, and are present in quantities sufficient to validate and confirm large-scale sequencing processes. Method validation may require known samples to confirm expected outcomes of the processes being validated. The inherent biological variability of biological samples obtained from individuals may not be suitable for the rigid requirements of process validation. The contrived biological samples described herein may approximate the complexity and biological features of a biological sample in a known and defined manner that may be controlled accordingly for the processes being validated.

### CROSS-REFERENCE

This application is a continuation of International Application No. PCT/US2023/068314, filed Jun. 12, 2023, which claims the benefit of U.S. Provisional Application No. 63/351,598, filed Jun. 13, 2022, each of which is incorporated by reference herein in its entirety.

### FIELD

The present disclosure relates generally to nucleic acid sequencing methods and reagents. More specifically, the present disclosure relates to artificial nucleic acid samples with known, predetermined characteristics and uses thereof, such as in process validation methods and controls for sequencing of nucleic acids in a biological sample.

### BACKGROUND

In the field of liquid biopsy for disease detection, cell-free nucleic acid (cfNA) may be an informative biological indicator of certain diseases and may be exploited in diagnostic tests. The process of scaling a diagnostic test from research phase to industrial development and broad commercial launch may require validation of reagents, methods, and processes. For diagnostic tests of cfDNA (e.g., cell-free DNA (cfDNA) or cell-free RNA (cfRNA)) in a biological sample, the industrial scale processes may need to be validated on cfNA-containing samples. Obtaining sufficient samples to assess and validate test performance may not be feasible, cost-prohibitive, and lacking in scientific controls.

Obtaining cfDNA samples from individuals with particular specific characteristics and in quantities large enough to perform industrial-scale process validation testing may be difficult if not impossible. Known, predetermined characteristics of such nucleic acid samples may include, for example, features such as nucleic acid length profiles, chemical profiles, or sequence profiles that resemble those in naturally-occurring biological samples, either from individuals or from groups of individuals.

Genome coverage may represent either all regions found in an original cfDNA sample or a subset of particular genomic regions of interest. These subset regions may be specifically selected, isolated, and amplified from either the original cfDNA sample, or a previously amplified cfDNA sample, through chemical, enzymatic, or physical methods. These contrived samples may be prepared with predetermined levels of nucleic acid modifications such that process accuracy may be ascertained in light of the predetermined level of nucleic acid modification.

Biological samples containing cfDNA may be highly valuable and expensive to obtain in sufficient quantities to use in process validation and control. Moreover, biological samples containing cfDNA may lack uniform and known qualities that are required to assess process validation and control. Certain artificially available nucleic acid samples (e.g., WGA HCT116 gDNA), may not be provided in the size range of cfDNA, and therefore may require additional pre-processing steps for suitability as process validation controls. For more specific process validation methods such as methylation analysis, available synthetic nucleic acid samples may lack known and defined nucleic acid methylation characteristics required, for example, for methylation sequencing. The lack of characteristics, such as appropriate size profile, fragment length, methylation profile, GC content, etc., may make these available samples unacceptable for cfDNA testing process validation without additional and costly pre-processing steps.

Recognizing the above-mentioned needs, the present disclosure provides contrived nucleic acid samples for process validation and control having specific, known, predetermined physical and chemical characteristics, and in quantities that are sufficient to validate and confirm large-scale sequencing processes.

### SUMMARY

The present disclosure provides methods and systems directed to contrived nucleic acid samples for process validation and control with predetermined, specific, and known physical and chemical characteristics, and in quantities that are sufficient to validate and confirm large-scale sequencing processes. Method validation requires known samples to confirm expected outcomes of the processes being validated. The inherent biological variability of biological samples obtained from individuals may not be suitable for the rigid requirements of process validation. The contrived biological samples described herein may approximate the complexity and biological features of a biological sample in a known and defined manner that may be controlled accordingly for the processes being validated. In contrast to other collections of nucleic acid fragments, such as expressed sequence tags (ESTs) or other cloned libraries, which are either fragments of the original molecules or are used in a final form that remain ligated to adapters or vectors necessary for function, the contrived biological samples described herein comprise nucleic acid fragment end points present in the original sample from which the contrived sample is produced.

In one aspect, provided herein is a method of preparing a contrived sample mixture of nucleic acid fragments, the method comprising:
obtaining a biological sample comprising a plurality of cell-free nucleic acids (cfNA);
subjecting the biological sample to nucleic acid amplification to produce a plurality of amplicons;
subjecting at least a portion of the plurality of amplicons to enzymatic digestion to produce a first mixture of nucleic acid fragments that has 0% of a predetermined nucleic acid chemical modification;
subjecting at least a portion of the plurality of amplicons to enzymatic digestion and a chemical reaction to produce a second mixture of nucleic acid fragments that has 100% of the predetermined nucleic acid chemical modification; and
mixing together a first quantity of the first mixture and a second quantity of the second mixture sufficient to produce the contrived sample mixture of nucleic acid fragments, wherein the contrived sample mixture of nucleic acid fragments has a predetermined level of the nucleic acid chemical modification.

In some embodiments, subjecting the biological sample to nucleic acid amplification is by polymerase chain reaction (PCR).

In some embodiments, the cfNA is cell-free DNA (cfDNA).

In some embodiments, each nucleic acid fragment of the contrived sample mixture has a fragment length of about 20-400 bp, about 50-300 bp, or about 100-250 bp.

In some embodiments, each nucleic acid fragment of the contrived sample mixture has a fragment length of about 50-400 bp, about 100-300 bp, about 120-220 bp, or about 167 bp.

In some embodiments, the contrived sample mixture comprises a predetermined total GC content that is known.

In some embodiments, the contrived sample mixture comprises a predetermined total GC content of between 0% and 100%.

In some embodiments, the contrived sample mixture comprises a predetermined total GC content of between 20% and 80%.

In some embodiments, each nucleic acid fragment of the contrived sample mixture has a fragment length and sequence end points of the cfNA of the biological sample.

In some embodiments, each nucleic acid fragment of the contrived sample mixture has a mononucleosome-sized fragment length, a dinucleosome-sized fragment length, a trinucleosome-sized fragment length, or a larger fragment length that is characteristic of the cfNA of the biological sample.

In some embodiments, each nucleic acid fragment of the contrived sample mixture has a mononucleosome-sized fragment length or a dinucleosome-sized fragment length that is characteristic of the cfNA of the biological sample.

In some embodiments, each nucleic acid fragment of the contrived sample mixture has a fragment length of about 168 bp, about 343 bp, about 533 bp, or about 2858 bp.

In some embodiments, each nucleic acid fragment of the contrived sample mixture is double stranded, wherein the cfNA of the biological sample is double stranded, wherein each nucleic acid fragment of the contrived sample mixture has the same 5′ sequence end points of both strands of the cfNA of the biological sample.

In some embodiments, the predetermined nucleic acid chemical modification comprises a methylation or a hydroxymethylation.

In some embodiments, the predetermined nucleic acid chemical modification comprises 5-methylcytosine (5mC) or 5-hydroxylmethylcytosine (5hmC).

In some embodiments, the predetermined nucleic acid chemical modification comprises 5mC, 5hmC, 5-formylcytosine (5fC), 5-carboxylcytosine (5caC), or a combination thereof.

In some embodiments, the predetermined level of the nucleic acid chemical modification is known.

In some embodiments, the predetermined level of the nucleic acid chemical modification is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the predetermined level of the nucleic acid chemical modification is about 0.001%, about 0.002%, about 0.003%, about 0.004%, about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, or about 0.02%.

In some embodiments, each nucleic acid fragment of the contrived sample mixture has a predetermined level of the nucleic acid chemical modification that is known.

In some embodiments, the predetermined level of the nucleic acid chemical modification in each of the nucleic acid fragments is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the predetermined nucleic acid chemical modification in each of the nucleic acid fragments is 5mC, 5hmC, 5-formylcytosine (5fC), 5-carboxylcytosine (5caC), or a combination thereof.

In some embodiments, the nucleic acid chemical modification for each base pair of each of the nucleic acid fragments is known.

In some embodiments, the contrived sample mixture and the cfNA in the biological sample have the same or substantially the same sequence profile.

In some embodiments, the sequence profile comprises a sequence size distribution, wherein the contrived sample mixture and the cfNA in the biological sample have the same or substantially the same sequence size distribution.

In some embodiments, the sequence size distribution is within a range that is less than about 500 bp.

In some embodiments, the sequence profile comprises coverage of all genomic regions in the cfNA in the biological sample.

In some embodiments, the sequence profile comprises coverage of a subset of all genomic regions in the cfNA in the biological sample.

In some embodiments, the subset of all genomic regions in the cfNA in the biological sample is a predetermined subset of genomic regions.

In some embodiments, the method further comprises suspending the contrived sample mixture in a biological medium. In some embodiments, the biological medium is serum, plasma, interstitial fluid, mucous, or an artificially-created equivalent thereof, wherein the biological medium is substantially free of nucleic acid molecules.

In one aspect, provided herein is a method of preparing a contrived sample mixture of nucleic acid fragments, the method comprising:
obtaining a biological sample comprising a plurality of cfNA;
subjecting the biological sample to nucleic acid amplification to produce a plurality of amplicons;
subjecting at least a portion of the plurality of amplicons to enzymatic digestion to produce a first mixture of nucleic acid fragments that has 0% methylated CpG sites;
subjecting at least a portion of the plurality of amplicons to enzymatic digestion and a methyltransferase reaction to produce a second mixture of nucleic acid fragments that has 100% methylated CpG sites; and
mixing together a first quantity of the first mixture and a second quantity of the second mixture sufficient to produce the contrived sample mixture of nucleic acid fragments, such that the contrived sample mixture of nucleic acid fragments has a pre-determined level of methylation of CpG sites.

In some embodiments, the method further comprises using the contrived sample mixture to validate a nucleic acid assay by:
processing the contrived sample mixture as a test assay to provide an expected assay performance based on a known contrived sample characteristic;
determining an actual assay performance of the test assay; and
identifying a difference between the actual assay performance and the expected assay performance for the contrived sample mixture,
wherein the nucleic acid assay is validated if the difference between the actual assay performance and the expected assay performance for the contrived sample mixture is within a predefined metric.

In some embodiments, the known contrived sample characteristic is a predetermined nucleic acid fragment size distribution, a predetermined level of the nucleic acid chemical modification, or a predetermined total GC content.

In some embodiments, the predefined metric comprises a total percentage of methylation in the contrived sample mixture.

In some embodiments, the predefined metric comprises accuracy, precision, specificity, linearity, detection limits, quantitation limits, or robustness.

In some embodiments, the method further comprises generating a validation report, wherein the validation report comprises results of the actual assay performance and the expected assay performance.

In some embodiments, the method further comprises:
processing the contrived sample mixture in a validated assay and determining assay performance of the mixture in the validated assay;
processing the contrived sample mixture in a unvalidated assay and determining assay performance of the contrived sample mixture in the unvalidated assay; and
identifying a difference between the validated assay performance and the unvalidated assay performance for the contrived sample mixture,
wherein the nucleic acid assay is validated if the difference between the validated assay performance and the unvalidated assay performance for the contrived sample mixture is within a predefined metric.

In some embodiments, the method further comprises generating a validation report, wherein the validation report comprises results of the validated test process performance and the unvalidated test process performance.

In some embodiments, the validated assay is a library preparation process, a chemical conversion process, a sequencing process, an amplification process, or a combination thereof.

In some embodiments, the chemical conversion process comprises chemical or enzymatic treatment of the nucleic acid fragments.

In some embodiments, the chemical conversion process comprises a methylation, an oxidation, a deamination, a fluoridation, a hydroxymethylation, a formylation, a glucosylation, an amination, or other nucleic acid modification that is enzymatically generated.

In some embodiments, the method further comprises determining a source of the difference between the actual assay performance and the expected assay performance for the contrived sample mixture.

In some embodiments, the source comprises an error in a library preparation process, a chemical conversion process, a sequencing process, an amplification process, or a combination thereof.

In some embodiments, the method further comprises distinguishing the error between two or more of the library preparation process, the chemical conversion process, the sequencing process, the amplification process, or the combination thereof.

In some embodiments, the method further comprises modifying one or more of the library preparation process, the chemical conversion process, the sequencing process, the amplification process, or the combination thereof, wherein the modifying is based at least in part on the error.

In one aspect, provided herein is a system of validating a predetermined sequencing process, the system comprising:
a validation database configured to store a set of assay validation metrics related to a nucleic acid assay and a contrived sample mixture used in the assay to provide an expected assay performance based on a known contrived sample characteristic; and
a computer processor configured to: i) assess performance characteristics of the assay, and ii) identify a difference between the performance characteristics of the assay and the set of process validation metrics,
wherein the nucleic acid assay is validated if the difference between the performance characteristics of the assay is within the set of process validation metrics.

In some embodiments, the known contrived sample characteristic is a predetermined nucleic acid fragment size distribution, a predetermined level of the nucleic acid chemical modification, or a predetermined total GC content.

In some embodiments, the computer processor is further configured to determine a source of the difference between the actual assay performance and the expected assay performance for the contrived sample mixture.

In some embodiments, the source comprises an error in a library preparation process, a chemical conversion process, a sequencing process, an amplification process, or a combination thereof.

In some embodiments, the computer processor is further configured to distinguish the error between two or more of the library preparation process, the chemical conversion process, the sequencing process, the amplification process, or the combination thereof.

In some embodiments, the computer processor is further configured to modify one or more of the library preparation process, the chemical conversion process, the sequencing process, the amplification process, or the combination thereof, wherein the modifying is based at least in part on the error.

In one aspect, provided herein is a contrived sample mixture comprising a plurality of nucleic acid fragments having a sequence length of between 20 bp and 400 bp, between 50 bp and 300 bp, or between 100 bp and 250 bp, wherein the contrived sample mixture comprises a predetermined total GC content of between 0% and 100%, and a predetermined total percentage of one or more nucleic acid chemical modifications.

In some embodiments, the predetermined total GC content is known.

In some embodiments, the predetermined total GC content is between 20% and 80%.

In some embodiments, each of the plurality of nucleic acid fragments has a fragment size and sequence end points of a cfNA of a biological sample from which the contrived sample mixture is derived.

In some embodiments, each of the plurality of nucleic acid fragments has a mononucleosome-sized fragment length, a dinucleosome-sized fragment length, a trinucleosome-sized fragment length, or a larger fragment length that is characteristic of a cfNA of a biological sample from which the contrived sample mixture is derived.

In some embodiments, each of the plurality of nucleic acid fragments has a fragment length of about 168 bp, about 343 bp, about 533 bp, or about 2858 bp.

In some embodiments, each of the plurality of nucleic acid fragments has a fragment size of a mononucleosome-sized fragment or a dinucleosome-sized fragment.

In some embodiments, each of the plurality of nucleic acid fragments has sequence end points of a cfNA of a biological sample from which the contrived sample mixture is derived.

In some embodiments, each of the plurality of nucleic acid fragments is double stranded, wherein a cfNA of a biological sample from which the contrived sample mixture is derived is double stranded, wherein each nucleic acid fragment of the contrived sample mixture has the same 5′ sequence end points of both strands of the cfNA of the biological sample.

In some embodiments, the one or more nucleic acid modifications comprise a methylation or a hydroxymethylation.

In some embodiments, the one or more nucleic acid modifications comprise 5-hydroxylmethylcytosine (5hmC) or 5-methylcytosine (5mC).

In some embodiments, the predetermined total percentage of the one or more nucleic acid chemical modifications is known.

In some embodiments, the predetermined total percentage of the one or more nucleic acid chemical modifications is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the one or more nucleic acid chemical modifications comprises a methylation modification selected from the group consisting of 5mC, 5hmC, 5fC, 5caC, and a combination thereof.

In some embodiments, a predetermined percentage of the methylation modification is about 0.001%, about 0.002%, about 0.003%, about 0.004%, about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, or about 0.02%.

In some embodiments, the total percentage of the one or more nucleic acid chemical modifications for each of the nucleic acid fragments is known.

In some embodiments, the total percentage of the one or more nucleic acid chemical modifications in each of the nucleic acid fragments is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the one or more nucleic acid chemical modifications comprises a methylation modification selected from the group consisting of 5mC, 5hmC, 5fC, 5caC, and a combination thereof.

In some embodiments, a predetermined percentage of the methylation modification is about 0.001%, about 0.002%, about 0.003%, about 0.004%, about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, or about 0.02%.

In some embodiments, the predetermined total percentage of the one or more nucleic acid chemical modifications for each of the plurality of nucleic acid fragments is known.

In some embodiments, the predetermined total percentage of the one or more nucleic acid chemical modifications in each of the plurality of nucleic acid fragments is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the one or more nucleic acid chemical modifications comprises a methylation modification selected from the group consisting of 5mC, 5hmC, 5fC, 5caC, and a combination thereof, wherein the one or more nucleic acid chemical modifications for each base pair of each of the nucleic acid fragments is known.

In some embodiments, each of the nucleic acid fragments has a fragment length of a cfNA of a biological sample from which the contrived sample mixture is derived.

In some embodiments, each of the nucleic acid fragments has a fragment length of between about 50-400 bp, between about 100-300 bp, between about 120-220 bp, or about 167 bp.

In some embodiments, a sequence profile of the contrived sample mixture has a sequence profile of a cfNA of a biological sample from which the contrived sample mixture is derived.

In some embodiments, a size distribution of a sequence profile of the contrived sample mixture is substantially similar to a size distribution of a sequence profile of a cfNA of a biological sample from which the contrived sample mixture is derived.

In some embodiments, the size distribution of the nucleic acid fragments is within a range that is less than about 500 bp.

In some embodiments, the sequence profile of the cfNA sample comprises coverage of all genomic regions in the cfNA.

In some embodiments, the sequence profile of the cfNA sample comprises coverage of a subset of all genomic regions in the cfNA.

In some embodiments, the subset of all genomic regions in the cfNA sample is a predetermined subset of genomic regions.

In some embodiments, the contrived sample mixture is suspended in a biological medium. In some embodiments, the biological medium is serum, plasma, interstitial fluid, mucous, or an artificially-created equivalent thereof, wherein the biological medium is substantially free of nucleic acid molecules.

In one aspect, provided herein is a mixture of nucleic acid fragments having known physical and chemical characteristics comprising:
sequence length of each fragment in the collection between 20 base pairs (bp) and 400 bp, 50 bp to 300 bp, 100 bp to 250 bp; predetermined GC content of the collection; predetermined nucleic acid chemical modifications; predetermined percentage of nucleic acid chemical modifications; or combinations thereof.

In some embodiments, the total GC content of nucleic acids in the mixture is known.

In some embodiments, the total GC content of the nucleic acids in the mixture is between 0% and 100%.

In some embodiments, the total GC content of the nucleic acids in the mixture is between 20% and 80%.

In some embodiments, the mixture of nucleic acid fragments comprises nucleic acid fragment size and sequence end points of a biological sample containing cell-free nucleic acid.

In some embodiments, the mixture of nucleic acid fragments comprises fragment lengths.

In some embodiments, the mixture of nucleic acid fragments comprises fragments having lengths corresponding to mono-nucleosomes (about 168 bp), di-nucleosomes (about 343 bp), and tri-nucleosomes (about 533 bp) and larger fragments (about 2858 bp) characteristic of a biological sample containing cell-free DNA.

In some embodiments, the mixture of nucleic acid fragments comprises nucleic acids having fragment size and sequence end points of sequences in the biological sample that is used to prepare the mixture of nucleic acid fragments.

In some embodiments, the contrived samples have fragment sizes approximating mononucleosome-sized and/or dinucleosome-sized nucleic acid fragments of biological samples comprising cell-free DNA.

In some embodiments, the contrived sample nucleic acid molecules comprise nucleic acid end points as the original biological sample used to prepare the contrived sample. In certain embodiments comprising double stranded DNA molecules, the contrived sample fragments may have the same 5′ end points of both original strands of a duplex nucleic acid fragment. In addition, the contrived sample fragments may have a known predetermined percentage of one or more nucleic acid modification. In one embodiment, the nucleic acid modification is a methylation or hydroxymethylation such as, but not limited to, 5-hydroxymethyl-cytosine or 5-methyl-cytosine. In other embodiments, the modification is selected from any nucleic acid modification that may be performed on a nucleic acid molecule in vitro.

The percentage of the modification (such as sequencing individual molecules) may be determined by using a single molecule test. A contrived sample may be confirmed because the individual strands would either have zero modification or have a very high amount of modification. The original samples, however, would have some low amount of modification across all of the molecules.

In some embodiments, the total percentage of nucleic acid chemical modification in the mixture is known.

In some embodiments, the total percentage of nucleic acid chemical modification in the mixture is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the nucleic acid chemical modification is a methylation modification selected from the group consisting of 5mC, 5hmC, 5fC, 5caC, and a combination thereof, wherein the total percentage modification for the nucleic acid sequences in the mixture is known.

In some embodiments, the total percentage of methylation modification in the mixture is about 0.001%, about 0.002%, about 0.003%, about 0.004%, about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, or about 0.02%.

In some embodiments, the predetermined total percentage of nucleic acid chemical modification in each of the plurality of nucleic acid fragments is known.

In some embodiments, the predetermined total percentage of nucleic acid chemical modification in each of the plurality of nucleic acid fragments is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In certain embodiments, the nucleic acid chemical modification is a methylation modification selected from the group consisting of 5mC, 5hmC, 5fC, 5caC, and a combination thereof, wherein the modification for each base pair in a nucleic acid sequence in the mixture is known.

In various embodiments, the contrived nucleic acid samples provide fragment length profiles matching biologically-obtained cfDNA samples, either from individuals, or from groups of individuals. In various embodiments, the fragment length profiles are between about 50-400 bp, about 100-300 bp, about 120-220 bp, or about 167 bp.

In various embodiments, the contrived DNA sequence profiles matching biological cfDNA samples, either from individuals, or from groups of individuals, DNA sequence profiles matching modified cfDNA samples, either from individuals, or from groups of individuals.

In various embodiments, the contrived sample nucleic acid sequence profiles are substantially similar to the size distribution of a nucleic acid sequence profiles of an original biological sample used to produce a contrived sample. In certain embodiments, the size profile of fragments in the sample is with a range less than about 500 bp. In certain embodiments, the fragment size profile is not identical to an original biological sample because the PCR rounds involved in producing a contrived sample can produce a selection bias for shorter molecules and for molecules with a more intermediate GC content. In certain embodiments, the fragment size profile of a contrived sample is smaller than an original biological sample used to produce the contrived sample, because the PCR rounds involved in producing a contrived sample can produce a selection bias for shorter molecules, and for molecules with a more intermediate GC content.

In various embodiments, the contrived nucleic acid sequence profiles comprise genome coverage representing substantially all regions found in a biological cfDNA sample, or a subset of particular predetermined genomic regions of interest.

In various embodiments, the predetermined subset of genomic regions may be specifically selected, isolated, and amplified from either the original cfDNA sample, or a previously amplified cfDNA sample, either through chemical, enzymatic, or physical methods.

In various embodiments, the mixture is suspended in a biological medium such as serum, plasma, interstitial fluid, mucous, or artificially-created equivalents that are free of nucleic acid molecules prior to addition of the mixture.

In certain embodiments, artificially-created biological media may comprise DNA

Depleted Human Plasma such as SBI, BioChain plasma, or Synthetic Plasma Substitute such as SigMatrix.

In an aspect, the present disclosure provides methods of validating a sequencing process comprising:

1. inputting a known contrived sample of nucleic acid fragments as a test process, wherein the contrived sample comprises a nucleic acid fragment distribution corresponding to cfDNA size distribution, known percentage sequence methylation, and known GC content to provide an expected test process accuracy and performance based on contrived sample characteristics;
2. assessing the process step accuracy and performance characteristics for the test process; and
3. identify differences between the test process and the expected process accuracy and performance for the known contrived sample,
wherein the process is validated if the differences between the performance of the test process and the expected process performance are within predefined metrics.

In certain embodiments, the predefined metrics are selected from metrics such as, for example, percentage of methylation in the contrived sample.

When the same contrived sample material is used in both a validated test process and an unvalidated test process (perhaps using different lots of reagents, or some other change), then the percentage methylation may be expected to be within a certain acceptable range (for example 1%) of what was determined in the validated process.

In various embodiments, the predetermined metrics are selected from the group consisting of accuracy, precision, specificity, linearity, detection limits, quantitation limits, and robustness.

In various embodiments, contrived samples may be used in processes where biological samples are scarce or have structural or chemical limitations that may confound process validation.

In various embodiments, the contrived sample performs in a similar or analogous manner or be commutable with biological samples. Commutability is determined by comparison of the measured result for a processed sample to the “scatter” of results for a representative set of samples measured using two measurement procedures.

In various embodiments, the contrived samples may be used in place of biological samples in the process or in combination with biological samples in the process to assess various features of the process during validation.

In one embodiment, the method provides a validation report describing results of the test sequencing process in comparison to the expected process parameters for the known contrived sample.

In various embodiments, the process is selected from the group consisting of a library preparation process, a chemical conversion process, a sequencing process, an amplification process, and a combination thereof.

The chemical conversion process may be a chemical or enzymatic treatment of the nucleic acid fragments in the contrived sample, or a combination thereof.

The chemical conversion may be a methylation, oxidation, deamination, fluoridation, hydroxymethylation, formylation, glucosylation, amination, or other base modifications that can be generated enzymatically.

In an aspect, the present disclosure provides a system of validating a predetermined sequencing process comprising:
a validation database configured to store a set of process validation metrics related to the at least one predetermined process and at least one predetermined contrived sample used in the process, wherein the contrived sample comprises a nucleic acid fragment distribution corresponding to cfDNA size distribution, known percentage sequence methylation, and known GC content to provide an expected test process accuracy and performance based on contrived sample characteristics; and
a computer processor configured to i) assess the process step accuracy and performance characteristics for the test process, and ii) identify differences between the test process and the process validation metrics for the predetermined process and contrived sample,
wherein the process is validated if the differences between the performance of the test process and the expected process performance are within predefined metrics.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

Examples of the present disclosure will now be described, by way of example only, with reference to the attached Figures. The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 provides an insert size histogram showing the size distribution of nucleic acid fragments in an exemplary contrived nucleic acid sample. The expected size distribution for cfDNA is about 167 bp for a mononucleosomal peak, and about 325 bp for a dinucleosomal peak.

FIG. 2 shows a graph depicting the average concentration of Post Extraction Contrived Samples after treatment with various conditions known to create environmental stress. The environmental stress conditions also relate to shipping and storage conditions for biological samples obtained for laboratory and clinical testing.

FIG. 3 shows methylation conversion rate of both control (biological cfDNA samples) and contrived cfDNA samples. PC, positive control (previously prepared and sequenced contrived material that demonstrated an HMF rate of 0). NTC, no template control.

FIG. 4 shows overall hypermethylated fragment (HMF) rate of control (biological cfDNA samples) and contrived cfDNA samples. PC, positive control (previously prepared and sequenced contrived material that demonstrated an HMF rate of 0). NTC, no template control.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### I. Mixtures of Nucleic Acids in Contrived Samples

The present disclosure relates generally to compositions, methods, and systems directed to contrived nucleic acid samples for process validation and control with prescribed specific and known physical and chemical characteristics, and in quantities that are sufficient to validate and confirm large-scale sequencing processes. Method validation may require known samples to confirm expected outcomes of the processes being validated. The inherent biological variability of biological samples obtained from individuals may not be suitable for the rigid requirements of process validation. The contrived biological samples described herein may approximate the complexity and biological features of a biological sample in a known and defined manner that may be controlled accordingly for the processes being validated.

As used herein, the term “contrived samples” generally refers to mixtures containing non-naturally-occurring nucleic acids having one or more known physical or chemical properties. In various embodiments, the contrived samples may be combined with physiological buffers to approximate a biological sample in need of process validation or other use thereof.

The various embodiments, the contrived samples comprise nucleic acid fragments with a desired size distribution range. In certain embodiments, the nucleic acid fragments may be obtained by digestion, amplification, enrichment with complementary probes, direct synthesis, or a combination thereof.

In one embodiment, the contrived sample comprises PCR-amplified cfDNA that is used as a DNA sample having the size distribution of genuine cfDNA samples.

In one embodiment, the cfDNA contrived sample comprises fragments having lengths corresponding to mononucleosomes (about 168 bp), dinucleosomes (about 343 bp), and trinucleosomes (about 533 bp), which may originate from cell lysis during apoptosis.

In one embodiment, the cfDNA contrived sample comprises larger fragments (about 2858 bp), which may originate from cell lysis during necrosis.

In one embodiment, the cfDNA contrived sample comprises more fragments reflecting nucleosome-sized fragments than fragments reflecting larger than nucleosome-sized fragments.

In one embodiment, the cfDNA contrived sample has the size distribution of cfDNA and lacks any of the DNA modifications found in biological DNA samples (for example, without any CpG methylation modifications).

In one embodiment, the cfDNA contrived sample has the size distribution of cfDNA and is highly modified to generate CpG methylation at most or all CpG sites.

In various embodiments, the CpG methylation in the nucleic acid sequences of the contrived sample is prepared by chemical or enzymatic conversion of the nucleic acid sample to obtain methylation at CpG sites. In another embodiment, the CpG methylation in the nucleic acid sequences of the contrived samples is prepared by direct synthesis with modified bases.

In one embodiment, the cfDNA contrived sample has the size distribution of cfDNA and contains a predetermined ratio of modified and unmodified CpG sites to provide precise levels of the desired modification for use. In one embodiment, a cfDNA contrived sample with a precise nucleic acid modification may be obtained by mixing a desired ratio of unmodified nucleic acid sequences and fully modified nucleic acid sequences.

In contrast to whole genome amplified genomic DNA or digested genomic DNA, the present contrived samples may be tightly regulated to possess predetermined size, structural, and chemical features, may be scalable for high volume use, and may be prepared easier and faster without additional steps for process validation in which cost is a significant factor.

In one embodiment, for use as an unmodified contrived “cfDNA” nucleic acid sample, the sample may be obtained from pooled cfDNA extracted from healthy plasma. The isolated biological cfDNA may then be subjected to a library preparation procedure to add amplification adapters. The nucleic acid may be subjected to 2 to 5 cycles of PCR using unmodified nucleobases to amplify the nucleic acid material. This amplification may effectively overwhelm any residual biological material in the sample that may contain modified bases. The resulting amplification product may be substantially free of modified bases and may comprises substantially 0% modified nucleic acid sample having the size distribution of cfDNA. Amplification adapters may be optionally cleaved by restriction digestion.

As used herein, the term “biological sample” refers to a sample of tissue, blood, serum, or lymph obtained from an individual to distinguish from a contrived sample that is prepared artificially to represent or model a biological sample for the various uses described herein.

As used herein, the term “biological cfDNA” refers to a sample of cell-free DNA isolated from a blood or tissue sample obtained from an individual that is processed to remove cells from the obtained sample in which the resulting processed cell-free sample contains DNA molecules.

In one embodiment, for use as a modified contrived “cfDNA” nucleic acid sample, the sample begins as pooled cfDNA extracted from healthy plasma. The isolated biological cfDNA may then be subjected to a library preparation procedure to add amplification adapters. The nucleic acid may then be subjected to conditions to modify bases in the sequence. The nucleic acid may be subjected to 2 to 5 cycles of PCR using unmodified nucleobases to amplify the nucleic acid material. This amplification may effectively overwhelm any residual biological material in the sample that may contain modified bases. The resulting amplification product may be substantially free of modified bases and may comprise a substantially 0% modified nucleic acid sample having the size distribution of cfDNA. Amplification adapters may be optionally cleaved by restriction digestion.

In certain embodiments, the modification is CpG site methylation and may be effected by chemical conversion, enzymatic conversion, or direct synthesis.

In one embodiment, for preparation methods using nucleic acid amplification, modified nucleobases are used in the amplification reaction to provide products having substantially fully-modified bases.

In other embodiments, a sample with a high level of modification may be used instead of a sample with substantially fully-modified bases. In various embodiments, a high level of modification may be greater than 50%, greater than 60%, greater than 70%, greater than 80%, greater than 90%, or greater than 95%. After a modified contrived sample is prepared, the amount of modification in the sample may be measured. In certain embodiments, a modified contrived sample may be used to set an arbitrary level of 1.0. The contrived sample may then be mixed with an unmodified sample having an arbitrary modification level set at 0.0 at certain ratios suitable for a desired level of modification. Contrived samples having controlled predetermined levels of modification may be prepared by mixing modified and unmodified contrived samples at ratios suitable for a desired level of nucleic acid modification.

In certain embodiments, ratios of substantially unmodified and high level of modified material may be prepared to provide contrived samples with controlled amounts of modified bases in the sample that are below the measured level of modification. For example, a modified sample created with 75% modified bases can be mixed with an unmodified sample in various ratios to create samples having predetermined levels of modification less than 75%.

In certain embodiments, the modification is CpG methylation. Such contrived samples may be useful for validating methylation sequencing workflow processes and methods. In certain embodiments, the arbitrary 0.0 level methylated sample is converted to methylate CpG cytosines in the nucleic acid molecules to provide an arbitrary 1.0 methylated sample using an enzymatic or chemical method, or a mixture of both. In one embodiment, the enzymatic method comprises reacting the sample with a methyltransferase enzyme. In certain embodiments, ratios of substantially 0% to substantially 100% CpG methylation material may be prepared to provide contrived samples with controlled amounts of CpG methylation in the sample.

The contrived samples may be prepared with predetermined levels of nucleic acid modifications such that process accuracy may be ascertained in light of the predetermined level of nucleic acid modification. In various embodiments, contrived samples are prepared having nucleic acid modifications levels of 0%, nucleic acid modifications at high levels, up to 100%, and samples with precise levels of nucleic acid modifications at any level between 0% and 100%. A high level of modification may be greater than 50%, greater than 60%, greater than 70%, greater than 80%, greater than 90%, or greater than 95%. In various embodiments, the nucleic acid modification is present at about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In certain embodiments, contrived sample material is prepared in which the modification is methylated cytosine bases (arbitrary levels 0.0 for unmodified and 1.0 modified). The unmodified and modified sample may be mixed at different ratios to mimic desired levels of methylation. In certain embodiments, a 0.1% mixture is used to represent high biological methylation, and a 0.01% is used to represent low biological methylation.

A contrived cfDNA sample may be mixed to a desired ratio of CpG methylation level and then spiked into a buffer such as BioChain Plasma. This mixing may be performed at different concentrations to obtain a predetermined desired nucleic acid concentration.

Advantages of the contrived samples described herein may include providing nucleic acid mixtures with defined characteristics for testing and validation applications in which the mixtures are produced faster, cheaper, and more abundantly than biological samples. For these applications, a small amount of biological cfDNA may be used with known reagents to prepare large volumes of contrived cfDNA in short time periods. In one example, milligrams of DNA material may be prepared in a few days to a week. Because biological cfDNA is used as a starting material for producing contrived samples, the prepared contrived sample may have the same size profile as cfDNA. This process may reduce the likelihood of the additional processing steps and unreliability of shearing DNA down to the correct size. In other embodiments, the prepared contrived samples may also be used as a control for quantitation methods, for example, using Fragment Analyzer or TapeStation systems, without having to waste precious (and often expensive) biological cfDNA.

Another advantage of using contrived samples is providing the user with flexibility to make various concentrations or percentage modifications of nucleic acid that may be required. In one embodiment for assessing methylation in cfDNA samples, contrived samples may be prepared having ranges from very high methylation (>75%) down to limits of detection (about or below 0.02% or 0.01%).

In one aspect, provided herein is a contrived sample mixture of nucleic acid fragments having known physical and chemical characteristics comprising:
sequence length of each fragment in the collection between 20 bp and 400 bp, 50 bp to 300 bp, 100 bp to 250 bp; predetermined GC content of the collection; predetermined nucleic acid chemical modifications; predetermined percentage of nucleic acid chemical modifications; or combinations thereof.

In certain embodiments, the total GC content of nucleic acids in the mixture is known.

In certain embodiments, the total GC content of the nucleic acids in the mixture is between 0% and 100%.

In certain embodiments, the total GC content of the nucleic acids in the mixture is between 20% and 80%.

In one embodiment, the mixture of nucleic acid fragments comprises nucleic acid fragment size and sequence end points of a biological sample containing cell-free nucleic acid.

In one embodiment, the mixture of nucleic acid fragments comprises fragment lengths

In one embodiment, the mixture of nucleic acid fragments comprises fragments having lengths corresponding to mononucleosomes (about 168 bp), dinucleosomes (about 343 bp), and trinucleosomes (about 533 bp), or larger cfDNA fragments (about 2858 bp) that are characteristic of a cell-free DNA of a biological sample from which the mixture is derived.

In certain embodiments, the total percentage of nucleic acid chemical modification in the mixture is known.

In certain embodiments, the total percentage of nucleic acid chemical modification in the mixture is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In certain embodiments, the nucleic acid chemical modification is a methylation modification selected from the group consisting of 5mC, 5hmC, 5fC, 5caC, and a combination thereof, wherein the total percentage modification for the nucleic acid sequences in the mixture is known.

In certain embodiments, the total percentage of methylation modification in the mixture is about 0.001%, about 0.002%, about 0.003%, about 0.004%, about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, or about 0.02%.

In certain embodiments, the total percentage of nucleic acid chemical modification in each nucleic acid fragment in the mixture is known.

In certain embodiments, the total percentage of nucleic acid chemical modification in each nucleic acid fragment in the mixture is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In certain embodiments, the nucleic acid chemical modification is a methylation modification selected from the group consisting of 5mC, 5hmC, 5fC, 5caC, and a combination thereof, wherein the modification for each base pair in a nucleic acid sequence in the mixture is known.

In various embodiments, the contrived nucleic acid samples provide fragment length profiles matching biologically-obtained cfDNA samples, either from individuals, or from groups of individuals. In various embodiments, the fragment length profiles are about 50-400 bp, about 100-300 bp, about 120-220 bp, or about 167 bp.

In various embodiments, the contrived nucleic acid samples provide fragment length profiles matching biologically-obtained cfDNA samples, either from individuals, or from groups of individuals. In various embodiments, the fragment length profiles are about 50-400 bp, about 100-300 bp, about 120-220 bp, or about 167 bp.

In various embodiments, the contrived DNA sequence profiles matching biological cfDNA samples, either from individuals, or from groups of individuals, DNA sequence profiles matching modified cfDNA samples, either from individuals, or from groups of individuals.

In various embodiments, the contrived sample nucleic acid sequence profiles are substantially similar to the size distribution of a nucleic acid sequence profiles of an original biological sample used to produce a contrived sample. In certain embodiments, the size profile of fragments in the sample is within a range less than about 500 bp. In certain embodiments, the fragment size profile is not identical to an original biological sample, because the PCR rounds involved in producing a contrived sample can produce a selection bias for shorter molecules, and for molecules with a more intermediate GC content. In certain embodiments, the fragment size profile of a contrived sample is lower than an original biological sample used to produce the contrived sample, because the PCR rounds involved in producing a contrived sample can produce a selection bias for shorter molecules, and for molecules with a more intermediate GC content.

With cfDNA samples, there is a strong 10 bp pattern of increased abundance (peaks), especially with fragments under 165 bp in length. In some individuals with high tumor fractions for example, these peaks can be highly increased in amount. In cases like this, given the profile of original samples, identifying from which original sample a contrived sample was generated may be possible.

In various embodiments, the contrived nucleic acid sequence profiles comprise genome coverage representing substantially all regions found in a biological cfDNA sample, or a subset of particular predetermined genomic regions of interest.

In various embodiments, the predetermined subset of genomic regions may be specifically selected, isolated, and amplified from either the original cfDNA sample, or a previously amplified cfDNA sample, either through chemical, enzymatic, or physical methods.

Many studies require contrived nucleic acid to be extracted from a matrix similar to the biological sample that a biologically-derived nucleic acid may be obtained from, using the same procedure used to isolate biologically-derived nucleic acid from the sample. For example, many studies require contrived cfDNA to be extracted from a matrix similar to blood plasma, using the same procedure used to isolate biologically-derived cfDNA samples from plasma. Contrived cfDNA may be extracted from a matrix to yield concentrations similar to genuine cfDNA from plasma.

In various embodiments, the mixture is suspended in a biological medium such as serum, plasma, interstitial fluid, mucous, or artificially-created medium that is substantially free of nucleic acid molecules prior to addition of the mixture.

In certain embodiments, artificially-created biological media include, but are not limited to, DNA Depleted Human Plasma such as SBI, BioChain plasma or Synthetic Plasma Substitute such as SigMatrix.

### II. Methods of Use

In certain embodiments, the contrived samples described herein are useful for simulating biological cfDNA with precise levels of DNA modification for testing, validating and defining processes and methods, when the biological samples having these levels of DNA modification are difficult or expensive to obtain. For example, testing the usage and limitations of an assay to detect a disease associated with a particular modification found in cfDNA.

In certain embodiments, the contrived samples described herein are useful for simulating biological DNA with precise levels of DNA modification for testing the quality of reagents used to identify levels of DNA modifications.

In certain embodiments, the contrived samples described herein are useful for producing large amounts of DNA samples with the particular size profiles, for example, size profiles of the cfDNA from particular individuals or groups of individuals, but with desired predetermined DNA modification levels.

In certain embodiments, the contrived samples may be used as nucleic acid process controls to analyze, confirm or validate processes such as nucleic acid extraction, fragment analyzer (such as TapeStation), chemical or enzymatic base conversion, PCR, library preparation, or a combination thereof.

In an aspect, the present disclosure provides methods of validating a sequencing process comprising:
inputting a known contrived sample of nucleic acid fragments as a test process, wherein the contrived sample comprises a nucleic acid fragment distribution corresponding to cfDNA size distribution, known percentage sequence methylation, and known GC content to provide an expected test process accuracy and performance based on contrived sample characteristics;
assessing the process step accuracy and performance characteristics for the test process, and
identify differences between the test process and the expected process accuracy and performance for the known contrived sample,
wherein the process is validated if the differences between the performance of the test process and the expected process performance are within predefined metrics.

In various embodiments, the predetermined metrics are selected from the group consisting of accuracy, precision, specificity, linearity, detection limits, quantitation limits, and robustness.

In various embodiments, the predetermined metrics for an enzymatic conversion process may be % nucleic recovery, nucleic acid yields, enzymatic conversion rate, modification percentage, or the like.

In various embodiments, contrived samples may be used in processes where biological samples are scarce or have structural or chemical limitations that may confound process validation.

In various embodiments, the contrived sample performs in a similar or analogous manner or be commutable with biological samples. Commutability is determined by comparison of the measured result for a processed sample to the “scatter” of results for a representative set of samples measured using two measurement procedures.

In various embodiments, the contrived samples may be used in place of biological samples in the process or in combination with biological samples in the process to assess various features of the process during validation.

In one embodiment, the method provides a validation report describing results of the test sequencing process in comparison to the expected process parameters for the known contrived sample.

In various embodiments, the process is selected from the group consisting of a library preparation process, a base conversion process, a sequencing process, an amplification process, and a combination thereof.

In various embodiments, the base conversion process is a chemical or enzymatic treatment of the nucleic acid fragments in the contrived sample, or a combination thereof.

In various embodiments, the base conversion is a methylation, oxidation, deamination, fluoridation, hydroxymethylation, formylation, glucosylation, amination, or another base modifications that can be generated enzymatically. In other embodiments, base modifications can be generated chemically, for example, but not limited to alkylation, and dimerization. In other embodiments, synthetic modifications can be introduced into the contrived samples using synthetic nucleotides and a mutant DNA polymerase. In certain embodiments, the modifications do not interfere with removal of an amplification adapter during preparation (e.g., by a nuclease).

### III. Systems

In combination with the contrived samples described herein, apparatuses, software, and interfaces may be used as a system to conduct methods described herein. Using apparatuses, software and interfaces, a user may enter, request, query or determine options for using particular information, programs or processes and validation thereof (e.g., sample extraction, nucleic acid purification, nucleic acid modification, library preparation, sequencing, mapping sequence reads, processing mapped data, quality control analysis and/or providing an outcome), which can involve implementing statistical analysis algorithms, statistical significance algorithms, statistical algorithms, iterative steps, validation algorithms, and graphical representations, for example. In some embodiments, a data set may be entered by a user as input information, a user may download one or more data sets by a suitable hardware media (e.g., flash drive), and/or a user may send a data set from one system to another for subsequent processing and/or providing an outcome (e.g., send sequence read data from a sequencer to a computer system for sequence read mapping; send mapped sequence data to a computer system for processing and yielding an outcome and/or report).

A system may comprise one or more apparatuses. Each apparatus comprises one or more of memory, one or more processors, and instructions. For a system that comprises two or more apparatus, some or all of the apparatus may be located at the same location, some or all of the apparatus may be located at different locations, all of the apparatus may be located at one location and/or all of the apparatus may be located at different locations. For a system that comprises two or more apparatus, some or all of the apparatus may be located at the same location as a user, some or all of the apparatus may be located at a location different than a user, all of the apparatus may be located at the same location as the user, and/or all of the apparatus may be located at one or more locations different than the user. In certain embodiments, the one or more apparatus may comprise centrifuges, thermocyclers, incubators, freezers, sequencers, and other apparatuses that may form part of a process in need of validation.

A system sometimes comprises a computing apparatus and a sequencing apparatus, where the sequencing apparatus is configured to receive physical nucleic acid and generate sequence reads, and the computing apparatus is configured to process the reads from the sequencing apparatus. The computing apparatus sometimes is configured to determine the presence or absence of a sample or process metric in need of validation.

A user may, for example, place a query to software which then may acquire a data set via internet access, and in certain embodiments, a programmable processor may be prompted to acquire a suitable data set based on given parameters. A programmable processor also may prompt a user to select one or more data set options selected by the processor based on given parameters. A programmable processor may prompt a user to select one or more data set options selected by the processor based on information found via the internet, other internal or external information, or the like. Options may be chosen for selecting one or more data feature selections, one or more statistical algorithms, one or more statistical analysis algorithms, one or more statistical significance algorithms, iterative steps, one or more validation algorithms, and one or more graphical representations of methods, apparatuses, or computer programs.

Systems addressed herein may comprise general components of computer systems, such as, for example, network servers, laptop systems, desktop systems, handheld systems, personal digital assistants, computing kiosks, and the like. A computer system may comprise one or more input means such as a keyboard, touch screen, mouse, voice recognition or other means to allow the user to enter data into the system. A system may further comprise one or more outputs, including, but not limited to, a display screen (e.g., CRT or LCD), speaker, FAX machine, printer (e.g., laser, ink jet, impact, black and white or color printer), or other output useful for providing visual, auditory and/or hardcopy output of information (e.g., outcome and/or report).

In a system, input and output means may be connected to a central processor which may comprise among other components, a microprocessor for executing program instructions and memory for storing program code and data. In some embodiments, processes may be implemented as a single user system located in a single geographical site. In certain embodiments, processes may be implemented as a multi-user system. In the case of a multi-user implementation, multiple central processors may be connected by means of a network. The network may be local, encompassing a single department in one portion of a building, an entire building, span multiple buildings, span a region, span an entire country, or be worldwide. The network may be private, being owned and controlled by a provider, or may be implemented as an internet based service where the user accesses a web page to enter and retrieve information. Accordingly, in certain embodiments, a system comprises one or more machines, which may be local or remote with respect to a user. More than one machine in one location or multiple locations may be accessed by a user, and data may be mapped and/or processed in series and/or in parallel. Thus, a suitable configuration and control may be utilized for mapping and/or processing data using multiple machines, such as in local network, remote network and/or “cloud” computing platforms.

A system can comprise a communications interface in some embodiments. A communications interface allows for transfer of software and data between a computer system and one or more external devices. Non-limiting examples of communications interfaces comprise a modem, a network interface (such as an Ethernet card), a communications port, a PCMCIA slot and card, and the like. Software and data transferred via a communications interface generally are in the form of signals, which can be electronic, electromagnetic, optical and/or other signals capable of being received by a communications interface. Signals may be provided to a communications interface via a channel. A channel often carries signals and can be implemented using wire or cable, fiber optics, a phone line, a cellular phone link, an RF link and/or other communications channels. Thus, in an example, a communications interface may be used to receive signal information that can be detected by a signal detection module.

Data may be input by a suitable device and/or method, including, but not limited to, manual input devices or direct data entry devices (DDEs). Non-limiting examples of manual devices include keyboards, concept keyboards, touch sensitive screens, light pens, mouse, tracker balls, joysticks, graphic tablets, scanners, digital cameras, video digitizers and voice recognition devices. Non-limiting examples of DDEs include bar code readers, magnetic strip codes, smart cards, magnetic ink character recognition, optical character recognition, optical mark recognition, and turnaround documents.

In some embodiments, output from a sequencing apparatus may serve as data that can be input via an input device. In certain embodiments, mapped sequence reads may serve as data that can be input via an input device. In certain embodiments, simulated data is generated by an in silico process and the simulated data serves as data that can be input via an input device. As used herein, the term “in silico” generally refers to research and experiments performed using a computer. In silico processes may include, but are not limited to, mapping sequence reads and processing mapped sequence reads according to processes described herein.

A system may include software useful for performing a process described herein, and software can comprise one or more modules for performing such processes (e.g., sequencing module, logic processing module, data display organization module). The term “software” refers to computer readable program instructions that, when executed by a computer, perform computer operations. Instructions executable by the one or more processors may be provided as executable code, that when executed, can cause one or more processors to implement a method described herein. A module described herein can exist as software, and instructions (e.g., processes, routines, subroutines) embodied in the software can be implemented or performed by a processor. For example, a module (e.g., a software module) can be a part of a program that performs a particular process or task. The term “module” refers to a self-contained functional unit that can be used in a larger apparatus or software system. A module can comprise a set of instructions for carrying out a function of the module. A module can transform data and/or information. Data and/or information can be in a suitable form. For example, data and/or information can be digital or analogue. In certain embodiments, data and/or information can be packets, bytes, characters, or bits. In some embodiments, data and/or information can be any gathered, assembled, or usable data or information. Non-limiting examples of data and/or information include a suitable media, pictures, video, sound (e.g., frequencies, audible or non-audible), numbers, constants, a value, objects, time, functions, instructions, maps, references, sequences, reads, mapped reads, levels, ranges, thresholds, signals, displays, representations, or transformations thereof. A module can accept or receive data and/or information, transform the data and/or information into a second form, and provide or transfer the second form to an apparatus, peripheral, component or another module. A module can perform one or more of the following non-limiting functions: mapping sequence reads, providing counts, assembling portions, providing or determining a level, providing a count profile, normalizing (e.g., normalizing sample measurements, sequence reads, normalizing counts, and the like), providing a normalized count profile or levels of normalized counts, comparing two or more levels, providing uncertainty values, providing or determining expected levels and expected ranges (e.g., expected level ranges, threshold ranges and threshold levels), providing adjustments to levels (e.g., adjusting a first level, adjusting a second level, adjusting a profile of a chromosome or a segment thereof, and/or padding), providing identification, categorizing, plotting, and/or determining an outcome, for example. A processor can, in certain embodiments, carry out the instructions in a module. In some embodiments, one or more processors are required to carry out instructions in a module or group of modules. A module can provide data and/or information to another module, apparatus, or source and can receive data and/or information from another module, apparatus, or source.

A computer program product sometimes is embodied on a tangible computer-readable medium, and sometimes is tangibly embodied on a non-transitory computer-readable medium. A module sometimes is stored on a computer readable medium (e.g., disk, drive) or in memory (e.g., random access memory). A module and processor capable of implementing instructions from a module can be located in an apparatus or in different apparatus. A module and/or processor capable of implementing an instruction for a module can be located in the same location as a user (e.g., local network) or in a different location from a user (e.g., remote network, cloud system). In embodiments in which a method is carried out in conjunction with two or more modules, the modules can be located in the same apparatus, one or more modules can be located in different apparatus in the same physical location, and one or more modules may be located in different apparatus in different physical locations.

An apparatus, in some embodiments, comprises at least one processor for carrying out the instructions in a module. Counts of sequence reads mapped to portions of a reference genome sometimes are accessed by a processor that executes instructions configured to carry out a method described herein. Counts that are accessed by a processor can be within memory of a system, and the counts can be accessed and placed into the memory of the system after they are obtained. In some embodiments, an apparatus comprises a processor (e.g., one or more processors) which processor can perform and/or implement one or more instructions (e.g., processes, routines and/or subroutines) from a module. In some embodiments, an apparatus comprises multiple processors, such as processors coordinated and working in parallel. In some embodiments, an apparatus operates with one or more external processors (e.g., an internal or external network, server, storage device and/or storage network, e.g., a cloud). In some embodiments, an apparatus comprises a module. In certain embodiments an apparatus comprises one or more modules. An apparatus comprising a module often can receive and transfer one or more of data and/or information to and from other modules. In certain embodiments, an apparatus comprises peripherals and/or components. In certain embodiments an apparatus can comprise one or more peripherals or components that can transfer data and/or information to and from other modules, peripherals and/or components. In certain embodiments an apparatus interacts with a peripheral and/or component that provides data and/or information. In certain embodiments peripherals and components assist an apparatus in carrying out a function or interact directly with a module. Non-limiting examples of peripherals and/or components include a suitable computer peripheral, I/O or storage method or device including but not limited to scanners, printers, displays (e.g., monitors, LED, LCT, or CRTs), cameras, microphones, pads (e.g., iPads, tablets), touch screens, smart phones, mobile phones, USB I/O devices, USB mass storage devices, keyboards, a computer mouse, digital pens, modems, hard drives, jump drives, flash drives, a processor, a server, CDs, DVDs, graphic cards, specialized I/O devices (e.g., sequencers, photo cells, photo multiplier tubes, optical readers, sensors, etc.), one or more flow cells, fluid handling components, network interface controllers, ROM, RAM, wireless transfer methods and devices (Bluetooth, WiFi, and the like), the world wide web (www), the internet, a computer, and/or another module.

Software may be provided on a program product containing program instructions recorded on a computer readable medium, including, but not limited to, magnetic media including floppy disks, hard disks, and magnetic tape; and optical media including CD-ROM discs, DVD discs, magneto-optical discs, flash drives, RAM, floppy discs, the like, and other such media on which the program instructions can be recorded. In online implementation, a server and web site maintained by an organization can be configured to provide software downloads to remote users, or remote users may access a remote system maintained by an organization to remotely access software. Software may obtain or receive input information. Software may comprise a module that specifically obtains or receives data (e.g., a data receiving module that receives sequence read data and/or mapped read data) and may comprise a module that specifically processes the data (e.g., a processing module that processes received data (e.g., filters, normalizes, provides an outcome and/or report).

As used herein, the terms “obtaining” and “receiving” input information generally refer to receiving data (e.g., sequence reads, mapped reads) by computer communication means from a local, or remote site, human data entry, or any other method of receiving data. The input information may be generated in the same location at which the input information is received. Alternatively, the input information may be generated in a different location and transmitted to the receiving location. In some embodiments, input information is modified before the input information is processed (e.g., placed into a format amenable to processing (e.g., tabulated).

Software can comprise one or more algorithms in certain embodiments. An algorithm may be used for processing data and/or providing an outcome or report according to a finite sequence of instructions. An algorithm often is a list of defined instructions for completing a task. Starting from an initial state, the instructions may describe a computation that proceeds through a defined series of successive states, eventually terminating in a final ending state. The transition from one state to the next is not necessarily deterministic (e.g., some algorithms incorporate randomness). By way of example, and without limitation, an algorithm can be a search algorithm, sorting algorithm, merge algorithm, numerical algorithm, graph algorithm, string algorithm, modeling algorithm, computational geometric algorithm, combinatorial algorithm, machine learning algorithm, cryptography algorithm, data compression algorithm, parsing algorithm, and the like. An algorithm can comprise one algorithm or two or more algorithms working in combination. An algorithm can be of any suitable complexity class and/or parameterized complexity. An algorithm can be used for calculation and/or data processing, and in some embodiments, can be used in a deterministic or probabilistic/predictive approach.

In certain embodiments, several algorithms may be implemented for use in software. These algorithms can be trained with raw data in some embodiments. For each new raw data sample, the trained algorithms may produce a representative processed data set or outcome. A processed data set sometimes is of reduced complexity compared to the parent data set that was processed. Based on a processed set, the performance of a trained algorithm may be assessed based on sensitivity and specificity. An algorithm with the highest sensitivity and/or specificity may be identified and utilized, in certain embodiments.

In certain embodiments, simulated (or simulation) data can aid data processing, for example, by training an algorithm or testing an algorithm. In some embodiments, simulated data comprises hypothetical various samplings of different groupings of sequence reads.

Simulated data may be based on what might be expected from a real population or may be skewed to test an algorithm and/or to assign a correct classification. Simulated data also is referred to herein as “virtual” data. Simulations can be performed by a computer program in certain embodiments. One possible step in using a simulated data set is to evaluate the confidence of an identified results, e.g., how well a random sampling matches or best represents the original data. One approach is to calculate a probability value (p-value), which estimates the probability of a random sample having a better score than the selected samples. In some embodiments, an empirical model may be assessed, assuming that at least one sample matches a reference sample (with or without resolved variations). In some embodiments, another distribution, e.g., Poisson distribution, may be used to define the probability distribution.

A system may comprise one or more processors in certain embodiments. A processor can be connected to a communication bus. A computer system may comprise a main memory, often random access memory (RAM), and can also comprise a secondary memory. Memory in some embodiments comprises a non-transitory computer-readable storage medium. Secondary memory can comprise, for example, a hard disk drive and/or a removable storage drive, representing a floppy disk drive, a magnetic tape drive, an optical disk drive, memory card and the like. A removable storage drive often reads from and/or writes to a removable storage unit. Non-limiting examples of removable storage units include a floppy disk, magnetic tape, optical disk, and the like, which can be read by and written to by, for example, a removable storage drive. A removable storage unit can comprise a computer-usable storage medium having stored therein computer software and/or data.

A processor may implement software in a system. In some embodiments, a processor may be programmed to automatically perform a task described herein that a user may perform. Accordingly, a processor, or algorithm conducted by such a processor, can require little to no supervision or input from a user (e.g., software may be programmed to implement a function automatically).

In some embodiments, one entity obtains blood from an individual, optionally isolates nucleic acid from the blood (e.g., from the plasma or serum), and transfers the blood or nucleic acid to a second apparatus that, incubates, amplifies, modifies, or generates sequence read data from the nucleic acid.

### EXAMPLES

### Example 1: a Contrived cfDNA Sample for Methylation Sequencing Process Validation

The sample preparation comprises obtaining cfDNA from multiple donors. The cfDNA is then processed through library preparation, PCR, and enzymatic digestion processes to produce a pool with cfDNA-like amplicons at 0% methylated CpG sites. A subset of the 0% methylated sample pool (0 methyl) is then subjected to a methyltransferase reaction to add methyl groups to all CpGs to create 100% methylated nucleic acid pool (1 methyl). The two contrived methylation pools (0 and 1 methylation) are then processed with the methylation sequencing process to determine the actual rate of methylated CpGs. Using the measured CpG methylation, the two pools are mixed to achieve desired CpG methylation ratios. Following formulation, each cfDNA mixture is spiked into a 50% mixture of PBST and DNA-depleted human plasma.

### Experimental Outline:

A suitable amount (e.g., 10 ng) of healthy pooled cfDNA is loaded in each well. End Repair and A-tailing of cfDNA (e.g., using an existing product/kit).

Ligation to amplification adapters containing a type II restriction site using a DNA ligase reaction:
a. Example sequences below for amplification adapters contain a BbsI-HF site
b. Adapters are at 50 μM concentration, use 2.5 μL/rxn

A suitable number of cycles (e.g., 10 cycles) of PCR amplification are performed. The resulting PCR product is then purified, the DNA is quantified, and the DNA is diluted.

A suitable number of additional cycles (e.g., 10 additional cycles) of PCR amplification are performed starting with a suitable amount (e.g., 10 ng) of the first PCR product. Alternatively, no additional cycles of PCR are performed.

Restriction digestion is performed to remove amplification adapters.

The DNA is then purified and then pooled for subsequent quantification.

A portion of this DNA is now modified (e.g., methylated using CpG Methyltransferase M.SssI).

Unmodified (“0%” or any other relatively low amount) and modified (“100%” or any other relatively high amount) DNA are mixed at various ratios.

The DNA mixture is then assayed:
c. Directly as purified DNA
d. Spiked into another matrix, such as real or artificial blood plasma

The contrived samples were robust and resistance to stress. The yields appeared to be robust after a freeze/thaw cycle and incubation at 37° C. for 1 hr.

All samples were prepared in nuclease-free BioChain Plasma and had an input of 30 ng. Yield improved with stress, although small amounts of DNA were detected in the plasma, which does not contribute to final metrics.

In order to detect small differences, the lower concentration was used. As observed in previous experiments, with a lower concentration of spike in there is a lower % recovery.

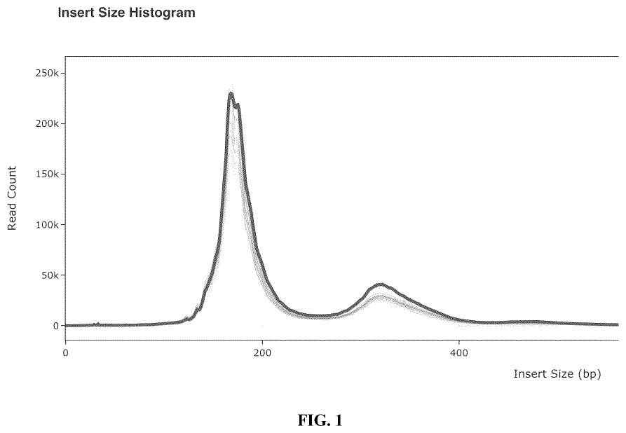

FIG. 1 provides an insert size histogram showing the size distribution of nucleic acid fragments in an example contrived nucleic acid sample. The expected size distribution for cfDNA is about 167 bp for a mononucleosomal peak and about 325 bp for a dinucleosomal peak.

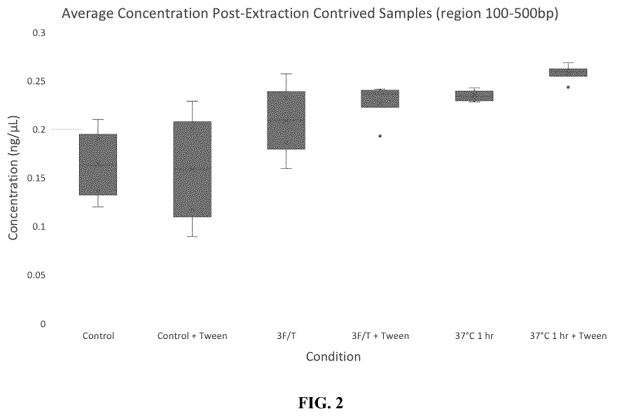

FIG. 2 shows a graph depicting the average concentration of Post Extraction Contrived Samples after treatment with various environmental stress conditions. The environmental stress conditions also relate to shipping and storage conditions for biological samples obtained for laboratory and clinical testing.

### Example 2: A Contrived cfDNA Sample For Methylation Sequencing Process Validation

Contrived samples were used to validate a methylation sequencing assay by distinguishing the source of hypermethylated fragment (HMF) rate “irregularities” between errors related to library preparation (or other assay steps) and low-level background “noise”. Noise may contribute to variability or error in measurement or modeling, and ultimately, incorrect product outputs. Noise may induce false positive or false negative calls.

Two categories of noise include: biological (e.g., variability across patients, samples, regions, etc.) and technical (e.g., measurement error in HMF rate due to wet-lab or computational processes). The identification of the source of background “noise” can drive the evaluation process of optimization options to reduce the noise. Different sources of noise may impact product output to different degrees and with different frequencies. Potential sources of the observation of methylated fragments in 0% contrived samples include: incomplete conversion, sample or batch contamination, residual methylation from the source donor cfDNA, random amplification (e.g., PCR) errors, alignment artifacts, etc.

The biological state must pass through the technical process, so technical noise may be layered on top of biological noise. Standard methods known in the art to separate noise sources may include:
Comparing patterns in biological replicates versus technical replicates
Measuring experimental steps early and often-the fewer steps before measurements, the fewer steps that can introduce noise
Using different assays with different technical artifacts
Using materials with known methylations states

In certain embodiments, contrived samples as disclosed herein may be used to distinguish false positive samples from true positive samples in model training/threshold-setting by determining the source of HMFs (biological or technical).

Experimental Design: Samples were prepared by obtaining cfDNA extracted from human plasma samples. Multiple aliquots of plasma were used to extract cfDNA from 5 healthy donors. Some sample material was set aside for methylation sequencing and some sample material was used to generate a matched 0% methylated contrived version. A sufficient amount of sample material was generated for both conditions to run all the samples in triplicate. Additional 0% methylated materials from pooled plasma samples were included to assess batch contamination and also serve as a positive control for the assay. The libraries were generated and then sequenced.

### Procedures:

Extraction: cfDNA was extracted from plasma or obtained directly from a previous assay.
Reagent Preparation
Library preparation
Sequencing of samples

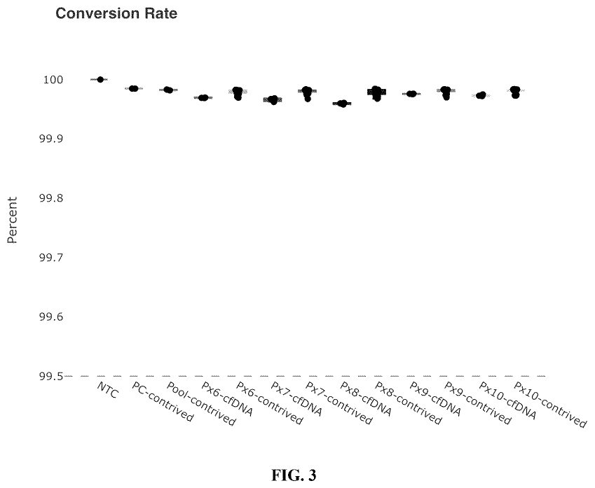

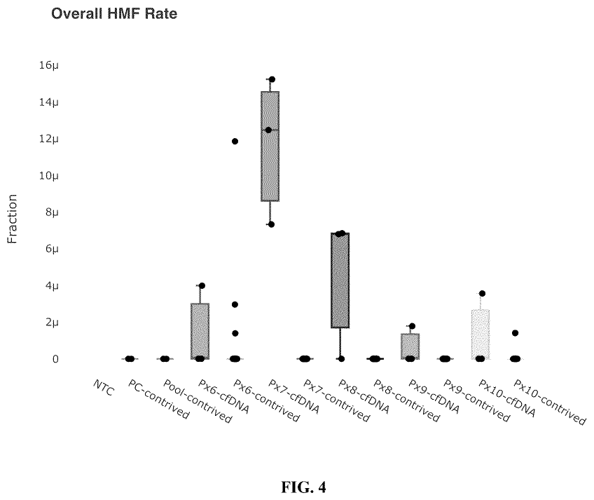

Results: The results of the validation assay are shown in FIG. 3 and FIG. 4.

FIG. 3 shows methylation conversion rate of control (biological cfDNA) and contrived cfDNA samples. Conversion rate was used as a quality control measure, where a higher conversion rate is understood to be better. Conversion rate quality control (QC) cut-off was 99.5%. All libraries satisfied QC and had a conversion rate >99.8%.

FIG. 4 shows overall hypermethylated fragment (HMF) rate of control (biological cfDNA samples) and contrived cfDNA samples. Non-zero HMF rates were observed in both biological and contrived cfDNA samples. HMF rate assesses the rate of occurrence of fragments with very high levels of methylation. Both biological and technical processes can lead to an increased presence of some number of HMFs in samples derived from healthy individuals. In this experiment, zero-percent methylation contrived material, generated from the cfDNA from healthy individuals, was expected to have an HMF rate of 0 (note: all contrived material presented in this experiment is 0% methylated).

To follow up on the potential source of HMFs in the biological cfDNA and contrived material in this experiment, the genomic location at which HMFs were observed was evaluated for each library. The genomic locations were sporadic in that they did not pattern with material type (biological cfDNA vs contrived), donor individual (i.e., not matched for cfDNA and contrived material originating from the same donor), or input mass into library preparation (data not shown).

### Conclusions:

The experiment demonstrated that technical noise in the assay was very low. HMFs in biological and contrived samples were broadly distributed across plasma donor and material type (biological cfDNA versus contrived sample).

HMFs did not appear to be the result of several potential sources of error in amplification, sequencing, or alignment.

HMFs from contrived samples were not residual original cfDNA molecules.

A slight leveling off in conversion rate with higher inputs was observed, but levels were not below 99.95%.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing an invention of the disclosure. It is therefore contemplated that the invention shall also cover any such alternatives, modifications, variations, or equivalents. It is intended that the following claims define the scope of the invention and that methods and structures within the scope of these claims and their equivalents be covered thereby.

1.-80. (canceled)

81. A method of preparing a contrived sample mixture of nucleic acid fragments, the method comprising:
(a) obtaining a biological sample comprising a plurality of cell-free nucleic acids (cfNA);
(b) subjecting the biological sample to nucleic acid amplification to produce a plurality of amplicons;
(c) subjecting at least a portion of the plurality of amplicons to enzymatic digestion to produce a first mixture of nucleic acid fragments that has substantially 0% of a predetermined nucleic acid chemical modification;
(d) subjecting at least a portion of the plurality of amplicons to enzymatic digestion and a chemical reaction to produce a second mixture of nucleic acid fragments that has substantially 100% of the predetermined nucleic acid chemical modification; and
(e) mixing a first quantity of the first mixture and a second quantity of the second mixture sufficient to produce the contrived sample mixture of nucleic acid fragments, wherein the contrived sample mixture of nucleic acid fragments has a predetermined level of the nucleic acid chemical modification.

82. The method of claim 81, wherein the nucleic acid amplification comprises polymerase chain reaction (PCR).

83. The method of claim 81, wherein the cfNA is cell-free deoxyribonucleic acid (cfDNA).

84. The method of claim 81, wherein each nucleic acid fragment of the contrived sample mixture has a fragment length of about 20 to about 400 nucleotides, about 50 to about 300 nucleotides, or about 100 to about 250 nucleotides.

85. The method of claim 81, wherein the contrived sample mixture comprises a predetermined total GC content of between 0% and 100%.

86. The method of claim 81, wherein the contrived sample mixture comprises a predetermined total GC content of between 20% and 80%.

87. The method of claim 81, wherein each nucleic acid fragment of the contrived sample mixture has a fragment length and sequence end points of the cfNA of the biological sample.

88. The method of claim 81, wherein each nucleic acid fragment of the contrived sample mixture has a mononucleosome-sized fragment length, a dinucleosome-sized fragment length, a trinucleosome-sized fragment length, or a larger fragment length that is characteristic of the cfNA of the biological sample.

89. The method of claim 81, wherein each nucleic acid fragment of the contrived sample mixture has a fragment length of about 168 nucleotides, about 343 nucleotides, about 533 nucleotides, or about 2858 nucleotides.

90. The method of claim 81, wherein each nucleic acid fragment of the contrived sample mixture is double stranded, wherein the cfNA of the biological sample is double stranded, wherein each nucleic acid fragment of the contrived sample mixture has the same 5′ sequence end points of both strands of the cfNA of the biological sample.

91. The method of claim 81, wherein the predetermined nucleic acid chemical modification comprises a methylation or a hydroxymethylation.

92. The method of claim 81, wherein the predetermined nucleic acid chemical modification comprises 5-methylcytosine (5mC), 5-hydroxylmethylcytosine (5hmC), 5-formylcytosine (5fC), 5-carboxylcytosine (5caC), or a combination thereof.

93. The method of claim 81, wherein the predetermined level of the nucleic acid chemical modification in each of the nucleic acid fragments is about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

94. The method of claim 81, wherein the contrived sample mixture and the cfNA in the biological sample have the same or substantially the same sequence profile.

95. The method of claim 94, wherein the sequence profile comprises a sequence size distribution, wherein the contrived sample mixture and the cfNA in the biological sample have the same or substantially the same sequence size distribution.

96. The method of claim 95, wherein the sequence size distribution is within a range that is less than about 500 nucleotides.

97. The method of claim 94, wherein the sequence profile comprises coverage of all genomic regions in the cfNA in the biological sample.

98. The method of claim 94, wherein the sequence profile comprises coverage of a subset of all genomic regions in the cfNA in the biological sample wherein the subset of all genomic regions in the cfNA in the biological sample is a predetermined subset of genomic regions.

99. The method of claim 94, further comprising suspending the contrived sample mixture in a biological medium.

100. The method of claim 99, wherein the biological medium is serum, plasma, interstitial fluid, mucous, or an artificially created equivalent thereof, wherein the biological medium is substantially free of nucleic acid molecules.

101. A method of preparing a contrived sample mixture of nucleic acid fragments, the method comprising:
(a) obtaining a biological sample comprising a plurality of cell-free nucleic acids (cfNA);
(b) subjecting the biological sample to nucleic acid amplification to produce a plurality of amplicons;
(c) subjecting at least a portion of the plurality of amplicons to enzymatic digestion to produce a first mixture of nucleic acid fragments that has 0% methylated CpG sites;
(d) subjecting at least a portion of the plurality of amplicons to enzymatic digestion and a methyltransferase reaction to produce a second mixture of nucleic acid fragments that has 100% methylated CpG sites; and
(e) mixing a first quantity of the first mixture and a second quantity of the second mixture sufficient to produce the contrived sample mixture of nucleic acid fragments, such that the contrived sample mixture of nucleic acid fragments has a pre-determined level of methylation of CpG sites.

102. A system of validating a predetermined sequencing process, the system comprising:
(a) a validation database configured to store a set of process validation metrics related to a nucleic acid assay and a contrived sample mixture used in the nucleic acid assay to provide an expected assay performance based on a known contrived sample characteristic; and
(b) a computer processor configured to: (i) assess performance characteristics of the nucleic acid assay, and (ii) identify a difference between the performance characteristics of the nucleic acid assay and the set of process validation metrics, wherein the nucleic acid assay is validated if the difference between the performance characteristics of the nucleic acid assay is within the set of process validation metrics.
